(19) United States
(12) Patent Application Publication
(10) Pub. No.: US 2023/0194542 A1
(43) Pub. Date: Jun. 22, 2023

(54) METHOD AND DEVICE FOR ANALYZING SIALIC-ACID-CONTAINING GLYCAN

(71) Applicant: SHIMADZU CORPORATION, Kyoto-Shi (JP)

(72) Inventor: Masaki Murase, Kyoto-Shi (JP)

(73) Assignee: SHIMADZU CORPORATION, Kyoto-Shi (JP)

(21) Appl. No.: 17/992,204

(22) Filed: Nov. 22, 2022

(30) Foreign Application Priority Data
Dec. 16, 2021 (JP) 2021-204045

(51) Int. Cl. G01N 33/68; G01N 27/62

(52) U.S. Cl. CPC G01N 33/6848; G01N 27/62; G01N 2440/38; G01N 2560/00

(57) ABSTRACT

Provided is a method for analyzing a sample containing a sialic-acid-containing glycan including a sialic-acid-linkage specific modification, based on mass spectrum data of the sample, including steps of: detecting, from the mass spectrum data, a representative peak for each isotope peak cluster; detecting, from the representative peaks, an isomer peak cluster including multiple ion peaks estimated to be identical in the number of sialic acids and the glycan composition exclusive of the sialic acids; estimating a glycan composition for each representative peak according to predetermined glycan search conditions; creating a mass spectrum with an annotation added for each isomer peak cluster to indicate a correspondence between each peak in one cluster and a peak in a mass spectrum, and displaying the annotated mass spectrum on a display section; and creating a table relating each estimated glycan-composition candidate to an isomer peak cluster, and displaying the table on the display section.

### TECHNICAL FIELD

The present invention relates to a method and device for analyzing a glycan by means of mass spectrometry, and more specifically, to an analysis method and analyzing device capable of an analysis of a sialic-acid-containing glycan, including the glycosidic linkage type of sialic acids. The term “glycan” in the present description includes not only a glycan in its independent form but also a form of a glycosylation, i.e., a glycan modifying a protein, peptide, lipid, nucleic acid or other kinds of biomolecules.

### BACKGROUND ART

An analysis of glycans is a major theme in bioscience, drug discovery, medicinal treatment and other related areas. Among other things, understanding the linkage type of sialic acids in a glycan which contains sialic acids is an important task in a glycan structure analysis. With such a technical background, techniques for a chemical modification specific to the linkage type of sialic acids have been developed in order to enable an efficient structural analysis of sialic-acid-containing glycans, including their difference in linkage type, by means of mass spectrometry. For example, Patent Literature 1 and Non Patent Literature 1 disclose a method for a sialic-acid-linkage specific modification, named SALSA (Sialic Acid Linkage-Specific Alkylamidation).

SALSA is a method which achieves isopropyl amidation and methyl amidation of α2,3- and α2,6-sialic acids, respectively, by utilizing the fact that the carboxylic acid in each of those sialic acids reacts in a different way when reacting with an amine to form an amide. This derivatization causes a mass difference of 28 Da between the α2,3- and α2,6-sialic acids, making it possible to distinguish between the α2,3- and α2,6-sialic acids based on the result of a mass spectrometric analysis.

Patent Literature 1 discloses a method for analyzing a glycan by using mass spectrum data obtained by a mass spectrometric analysis in which the SALSA method is employed as a pretreatment method. In the analysis method described in the document, three peaks located at intervals of 28 Da in a mass spectrum are detected as sialic-acid-linkage isomer ion peaks originating from sialic-acid-containing glycans, and an exhaustive search, with the kinds and numbers of monosaccharides as the search conditions, is performed for those peaks to estimate the glycan composition. From among the glycan-composition candidates obtained by the estimation, a composition which includes two or more α2,6-linkages is extracted as a highly plausible composition candidate for a peak which contains two or more sialic acids and shows the largest mass-to-charge ratio. The result can be displayed in the form of a table in which that highly plausible composition candidate is visually discriminated from the other composition candidates which are rather implausible to be sialic-acid-containing glycan isomers (for example, see FIGS. 6 and 8 in Patent Literature 1).

### CITATION LIST

### Patent Literature

Patent Literature 1: WO 2017/145496 A

### NON PATENT LITERATURE

Non Patent Literature 1: Takashi Nishikaze and five other authors, “Differentiation of Sialyl Linkage Isomers by One-Pot Sialic Acid Derivatization for Mass Spectrometry-Based Glycan Profiling”, Analytical Chemistry, 2017, Vol. 89, pp. 2353-2360

### SUMMARY OF INVENTION

### Technical Problem

As a matter of course, in the case of the mass spectrometric analysis of a sample on which a linkage-specific modification has been performed by SALSA or similar methods, the number of peaks observed in the mass spectrum will be larger than in the case of the mass spectrometric analysis of the same sample with no such pretreatment performed. Therefore, when a plurality of different kinds of sialic-acid-containing glycans which are different in glycan composition (or other aspects) are present in the sample, or furthermore, when a plurality of sialic-acid-containing glycans which are identical in the number of sialic acids and in the glycan structure exclusive of the sialic acids yet different in the kinds of sialic acids are present in the sample, a considerably large number of peaks will be observed in the mass spectrum, so that the peaks of isomer ions originating from different kinds of sialic-acid-containing glycans may possibly be observed in a mixed form.

In that case, the number of composition candidates shown in the composition candidate table as composition candidates of the sialic-acid-containing glycans will also be extremely large. The composition candidate table of shows the estimated glycan-composition candidates arranged in order of the m/z values of the original peaks. When there is an extremely large number of peaks in the mass spectrum for the previously described reason and the mass spectrum is complex, it will be difficult to recognize the correspondence between the peaks in the mass spectrum and the glycan-composition candidates in the composition candidate table. This causes problems for a user (i.e., an individual in charge of the data analysis): for example, it will be difficult to understand which glycan-composition candidates in the composition candidate table correspond to a plurality of peaks which are spaced at intervals of 28 Da and estimated to belong to one isomer peak cluster in the mass spectrum. As another example, if there is a peak having a plurality of glycan-composition candidates shown in the composition candidate table, it will be difficult to recognize glycan-composition candidates of another peak which is estimated to belong to the same isomer peak cluster as the peak in question. These problems are likely to lower the efficiency of the analyzing task by the user, causing the user to require a longer period of time for the analyzing task or make some errors, such as mistaking one peak for another or overlooking an important peak.

The present invention has been developed to solve these problems. Its primary objective is to provide a method and device for analyzing a sialic-acid-containing glycan which can improve the efficiency of the task of analyzing sialic-acid-containing glycans and can also enhance the analysis accuracy by lowering the probability of manual errors.

### Solution to Problem

One mode of the method for analyzing a sialic-acid-containing glycan according to the present invention developed for solving the previously described problem is an analysis method for analyzing a sample containing a sialic-acid-containing glycan including a modification specific to a sialic-acid linkage type, or a sample containing a molecule modified with the same glycan, based on mass spectrum data obtained by a mass spectrometric analysis of the sample, the method including:

a peak detection step for detecting, from the mass spectrum data, a representative peak for each isotope peak cluster;

a peak cluster detection step for detecting, from representative peaks detected in the peak detection step, an isomer peak cluster including a plurality of ion peaks estimated to be identical in the number of sialic acids and in the glycan composition exclusive of the sialic acids;

a composition estimation step for estimating a glycan composition for a representative peak detected in the peak detection step, according to a predetermined glycan search condition;

a first display process step for creating an annotated mass spectrum in which an annotation is added for each isomer peak cluster detected in the peak cluster detection step to indicate the correspondence between each peak included in one isomer peak cluster and a peak observed in a mass spectrum, or creating a peak table which shows, for each isomer peak cluster detected in the peak cluster detection step, the mass-to-charge-ratio values of the peaks included in one isomer peak cluster, and for displaying the mass spectrum or the peak table on a display section; and

a second display process step for creating a composition candidate table in which each glycan-composition candidate obtained in the composition estimation step is related to at least one isomer peak cluster detected in the peak cluster detection step, and for displaying the composition candidate table on the display section along with or in a switchable manner with the annotated mass spectrum or the peak table.

One mode of the device for analyzing a sialic-acid-containing glycan according to the present invention developed for solving the previously described problem is an analyzing device for analyzing a sample containing a sialic-acid-containing glycan including a modification specific to a sialic-acid linkage type, or a sample containing a molecule modified with the same glycan, based on mass spectrum data obtained by a mass spectrometric analysis of the sample, the device including:

a peak detector configured to detect, from the mass spectrum data, a representative peak for each isotope peak cluster;

a peak cluster detector configured to detect, from representative peaks detected by the peak detector, an isomer peak cluster including a plurality of ion peaks estimated to be identical in the number of sialic acids and in the glycan composition exclusive of the sialic acids;

a composition estimator configured to estimate a glycan composition for a representative peak detected by the peak detector, according to a predetermined glycan search condition;

a display processor configured to create an annotated mass spectrum in which an annotation is added for each isomer peak cluster detected by the peak cluster detector to indicate the correspondence between each peak included in one isomer peak cluster and a peak observed in a mass spectrum, or to create a peak table which shows, for each isomer peak cluster detected by the peak cluster detector, the mass-to-charge-ratio values of the peaks included in one isomer peak cluster, and further configured to create a composition candidate table in which each glycan-composition candidate obtained by the composition estimator is related to at least one isomer peak cluster detected by the peak cluster detector, and to display the composition candidate table on a display section along with or in a switchable manner with the annotated mass spectrum or the peak table.

### Advantageous Effects of Invention

In the previously described modes of the method and device for analyzing a sialic-acid-containing glycan according to the present invention, the user can intuitively recognize a plurality of peaks included in each isomer peak cluster by referring to the annotated mass spectrum or peak table shown on the display section. Subsequently, the user can refer to the composition candidate table and easily recognize, for each of the peaks included in one isomer peak cluster, the glycan-composition candidates corresponding to the peak, and determine, for example, a precursor ion for an MS/MS analysis necessary for determining which of the listed candidates is the most plausible candidate.

Thus, the present invention can improve the efficiency of the task of analyzing sialic-acid-containing glycans, including the linkage type of sialic acids. It can also lower the probability of manual errors in the analyzing task, thereby enhancing the analysis accuracy.

### BRIEF DESCRIPTION OF DRAWINGS

FIG. 1 is a block configuration diagram of one embodiment of a glycan analyzing system including a device for analyzing a sialic-acid-containing glycan according to the present invention.

FIG. 2 is a flowchart showing the procedure of the data-analyzing process in the glycan analyzing system according to the present embodiment.

FIG. 3 is a diagram showing one example of the annotated mass spectrum displayed in the glycan analyzing system according to the present embodiment.

FIG. 4 is a table showing one example of the peak table corresponding to the annotated mass spectrum shown in FIG. 3.

FIG. 5 is a table showing one example of the glycan-composition candidate table for a portion of the peaks in the peak table shown in FIG. 4.

### DESCRIPTION OF EMBODIMENTS

In the present invention, a “molecule modified with a sialic-acid-containing glycan” is, for example, a biomolecule, such as a protein, peptide, lipid or nucleic acid, modified with a sialic-acid-containing glycan.

A “modification specific to a sialic-acid linkage type”, on which the present invention is premised, is typically a SALSA method disclosed in Patent Literature 1 or Non Patent Literature 1 mentioned earlier, but is not limited to this method. It may be any method which causes a sialic-acid-linkage specific chemical modification (derivatization) that allows for the discrimination of two or more different linkage types of sialic acids, such as the α2,3-linkage, α2,6-linkage and α2,8-linkage, by mass difference.

There is no specific limitation on the type of mass spectrometer for performing a mass spectrometric analysis on a sample containing a sialic-acid-containing glycan (and other compounds). Example of the available mass spectrometers include ion trap mass spectrometers, linear ion trap mass spectrometers, TOF/TOF mass spectrometers, quadrupole time-of-flight (Q-TOF) mass spectrometers, quadrupole ion trap mass spectrometers, and Fourier-transform ion cyclotron resonance mass spectrometers.

One embodiment of a glycan analyzing system including an analyzing device for carrying out the method for analyzing a sialic-acid-containing glycan according to the present invention is hereinafter described referring to the attached drawings.

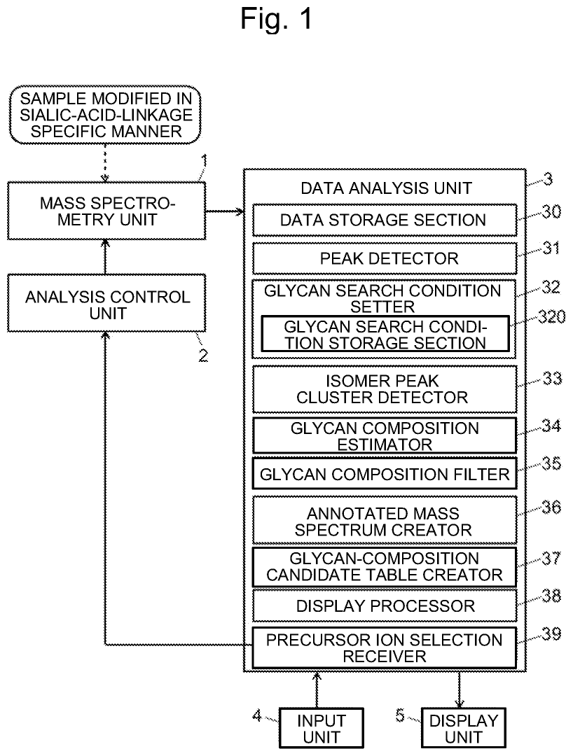

FIG. 1 is a schematic block configuration diagram of the present glycan analyzing system.

As shown in FIG. 1, the present system includes: a mass spectrometry unit 1 configured to perform a mass spectrometric analysis on a sample, an analysis control unit 2 configured to control the mass spectrometry unit 1, a data analysis unit 3 configured to perform an analyzing process on data obtained by a mass spectrometric analysis, as well as an input unit 4 and a display unit 5 serving as a user interface.

The data analysis unit 3 includes, as its functional blocks, a data storage section 30, peak detector 31, glycan search condition setter 32, isomer peak cluster detector 33, glycan composition estimator 34, glycan composition filter 35, annotated mass spectrum creator 36, glycan-composition candidate table creator 37, display processor 38 and precursor ion selection receiver 39. The glycan search condition setter 32 includes a glycan search condition storage section 320 as a sub-functional block.

There is basically no limitation on the type of mass spectrometry unit 1, although a mass spectrometer having an ion trap, collision cell or similar device capable of fragmenting ions by collision induced dissociation (CID) or other appropriate methods should be used in the case where an MS/MS analysis is carried out, as will be described later.

The mass spectrometry unit 1 does not need to be a mass spectrometer in an independent form; a liquid chromatograph mass spectrometer (LC-MS) may also be used. The unit may also be a system in which a plurality of samples are prepared by preparative separation and fractionation of an eluate containing components separated from each other by a liquid chromatograph, and those samples are individually subjected to a mass spectrometric analysis by a mass spectrometer.

The data analysis unit 3 in the present system is actually a personal computer or more sophisticated workstation, on which the functions of the functional blocks shown in FIG. 1 can be realized by executing, on the computer, a dedicated data processing program installed on the same computer. In that case, the input unit 4 is a keyboard and a pointing device (e.g., mouse) provided for the computer, while the display unit 5 is a monitor provided for the same computer.

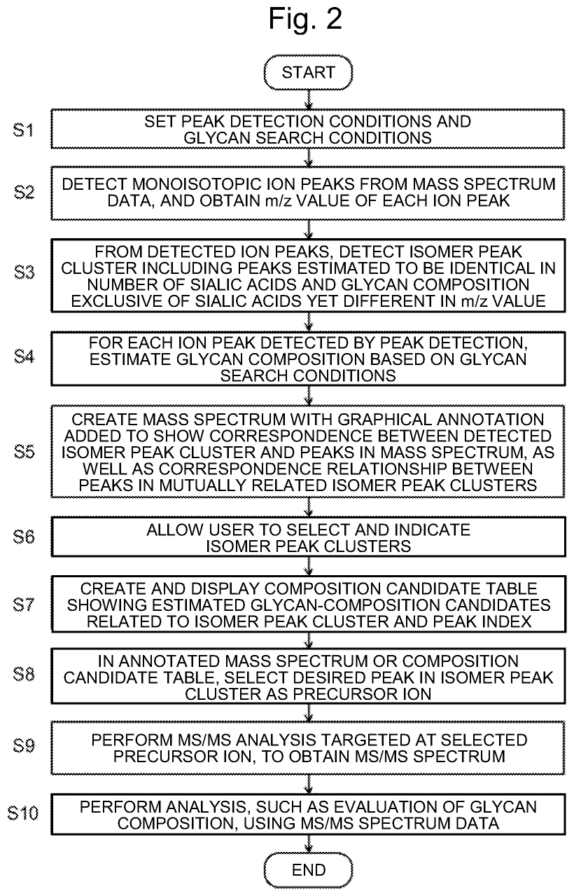

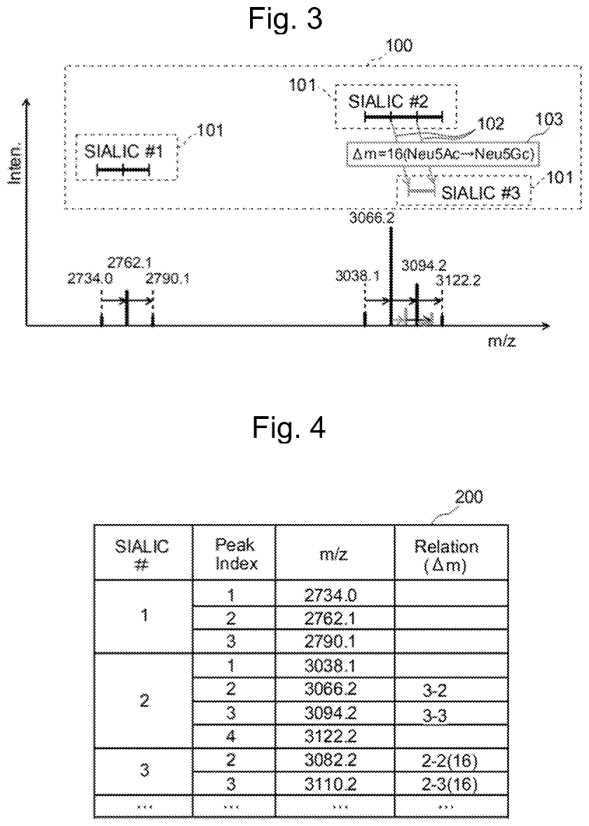

A procedure of the analysis of a sialic-acid-containing glycan in the glycan analyzing system according to the present embodiment is hereinafter described, referring to FIGS. 2-5 and including descriptions of an experimental example. FIG. 2 is a flowchart showing the procedure of the glycan analysis mainly carried out by the data analysis unit 3. FIGS. 3-5 are examples of the graph and tables to be displayed on the display unit 5 in the course of the analytical processing.

For an analysis of a sialic-acid-containing glycan by the glycan analyzing system according to the present embodiment, a pretreatment by a sialic-acid-linkage specific chemical modification is performed on a sample containing a molecule (such as a glycopeptide or glycolipid) which contains or is modified with a sialic-acid-containing glycan. The pretreated sample is subjected to a mass spectrometric analysis in the mass spectrometry unit 1. As for the method for the sialic-acid-linkage specific modification, for example, the SALSA method described in Non Patent Literature 1 (or other related documents) can be used. As explained earlier, two molecules which are identical in glycan composition exclusive of the sialic acids will have a mass difference of 28 Da after they have been modified by the SALSA method if a sialic acid contained in the glycan is the α2,3-linkage type in one molecule and the α2,6-linkage type in the other. A set of mass spectrum data covering a predetermined m/z range acquired by the mass spectrometric analysis is sent from the mass spectrometry unit 1 to the data analysis unit 3 and stored in the data storage section 30.

When the analytical processing based on mass spectrum data has been initiated, the glycan search condition setter 32 displays a glycan search condition setting window on the screen of the display unit 5 and prompts the user to enter glycan search conditions (Step S1). It is also possible to automatically use a default setting of the glycan search conditions, instead of requiring entry by the user. In addition to the glycan search conditions, peak detection conditions for the detection of the peaks from mass spectrum data may also be included in the setting window for the entry by the user. The entered or default settings of the glycan search conditions and peak detection conditions are stored in the glycan search condition storage section 320.

The glycan search conditions may include, for example, the sialic-acid-linkage specific modification method to be used, the allowable mass accuracy for the glycan composition estimation, the assumed ion species, as well as the kinds and numbers of sugar residues to be searched for (including sialic acids). The peak detection conditions may include, for example, the signal intensity or signal-to-noise ratio to be used as the threshold for recognizing a peak.

When a substantive analysis is initiated, the peak detector 31 retrieves the mass spectrum data to be analyzed from the data storage section 30 and detects a monoisotopic ion peak according to the peak detection conditions as the representative peak of each isotope peak cluster. In general, in the case of a molecule derived from a living organism, like glycans, a peak having the smallest m/z value among the plurality of isotopic ion peaks which appear, for example, at intervals of 1 Da can be detected as the monoisotopic ion peak. The m/z value of each detected ion peak is determined, and a peak list is created (Step S2). It is also possible to calculate an average (centroid) of the m/z values of the plurality of isotopic ion peaks in place of the m/z value of the monoisotopic ion peak. In summary, what is required is to determine an m/z value representative of a cluster for each of the isotopic ion peak clusters originating from the same kind of glycan.

In an experimental example performed by the present inventor, an N-linked glycan which modifies fetuin, which is a kind of protein in the blood of a fetal calf, was cleaved by PNGase, which is a deglycosylation enzyme, and the resulting fragments were enriched to obtain a glycan mixture as a specimen. The sialic acids contained in that specimen were subsequently modified by the SALSA method in a sialic-acid-linkage specific manner, and the reducing terminal of the glycans was labelled with anthranilic acid to obtain a sample for the analysis. This labelling is a pretreatment for promoting the ionization in a negative ion mode. A mass spectrometric analysis of the sample obtained by the pretreatment was performed by a matrix assisted laser desorption/ionization ion trap time-of-flight mass spectrometer (MALDI-IT-TOFMS) in a negative ion mode to obtain mass spectrum data.

In the experimental example, the glycan search conditions were set as follows.

Sialic-acid-linkage specific modification method: SALSA method

Allowable mass accuracy for glycan composition estimation: m/z 0.2

Ion species: deprotonated ion

Kinds and numbers of sugar residues to be searched for: Hexose, 3-15; HexNAc, 2-14; fucose (dHex), 0-2; Neu5Ac (sialic acid), 0-5; and Neu5Gc (sialic acid), 0-5.

The mass spectrum shown in FIG. 3 consists of only the monoisotopic ion peaks detected in Step S2, extracted from the mass spectrum data obtained by the mass spectrometric analysis in the experimental example.

Next, the isomer peak cluster detector 33 detects, from the peaks included in the peak list created in Step S2, an isomer peak cluster including a plurality of peaks which are estimated to be identical in the number of sialic acids and in the glycan composition exclusive of the sialic acids (Step S3).

A specific procedure is as follows: In the present example, the SALSA method is used in the pretreatment for the sialic-acid-linkage specific modification. Therefore, according to the SALSA method, the isomer peak cluster detector 33 detects, from the peak list, each group of ion peaks adjacent to each other at intervals of 28 Da which equals the difference in m/z between the α2,6-sialic acid and the α2,3-sialic acid. Each of the detected groups is assumed to be an isomer peak cluster consisting of linkage isomers of the sialic-acid-containing glycans which are identical in the number of sialic acids and in the glycan composition exclusive of the sialic acids. The allowable error of the peak interval used for the detection may be m/z 0.1, for example. Understandably, the number of ion peaks included in one isomer peak cluster changes depending on the number of sialic acids contained in the sialic-acid-containing glycan, linkage type of sialic acids and other factors.

In the mass spectrum shown in FIG. 3, three clusters of isomer peaks, all of which originate from singly charged ions, have been detected: The first isomer peak cluster (SIALIC #1) includes three peaks with m/z values of m/z 2734.0, m/z 2762.1 and m/z 2790.1. The second isomer peak cluster (SIALIC #2) includes four peaks with m/z values of m/z 3038.1, m/z 3066.2, m/z 3094.2 and m/z 3122.2. The third isomer peak cluster (SIALIC #3) includes two peaks with m/z values of m/z 3082.2 and m/z 3110.2.

Among those isomer peak clusters, some of the peaks in the second isomer peak cluster SIALIC #2 are distanced from some of the peaks in the third isomer peak cluster SIALIC #3 by 16 Da, which corresponds to the mass difference between N-acetylneuraminic acid (Neu5Ac) and N-glycolylneuraminic acid (Neu5Gc). This fact suggests the possibility that the third isomer peak cluster SIALIC #3 has glycan compositions which have resulted from the replacement of N-acetylneuraminic acid by N-glycolylneuraminic acid in the ions corresponding to the peaks in the second isomer peak cluster SIALIC #2.

Subsequently, the glycan composition estimator 34 estimates the glycan composition of each peak included in the peak list created in Step S2, according to the glycan search conditions stored in the glycan search condition storage section 320, and determines glycan-composition candidates (Step S4). Specifically, it performs an exhaustive search for the glycan compositions which match the m/z values of the ion peaks within the predetermined allowable mass accuracy under the specified conditions of the numbers and kinds of sugar residues. A successful search for the glycan-composition candidates does not always end with the candidates narrowed down to one; there may be two or more candidates ultimately obtained.

Subsequently, the annotated mass spectrum creator 36 creates a mass spectrum with a graphical annotation added to relate each peak in the isomer peak cluster detected in Step S3 to the corresponding peak observed in the mass spectrum. The display processor 38 shows the annotated mass spectrum on the screen of the display unit 5 (Step S5).

In FIG. 3, the graphical annotation 100 is added to the aforementioned mass spectrum which shows only the monoisotopic ion peaks. As shown in FIG. 3, the graphical annotation 100 includes a cluster indication mark 101 consisting of a horizontal bar which covers an m/z range from the smallest m/z value and the largest m/z value of a plurality of peaks included in one isomer peak cluster, and vertical bars each of which corresponds to the position of the m/z value of one of those peaks. From the number of vertical bars in this cluster indication mark 101, the user can intuitively recognize the number of peaks forming the isomer peak cluster, which is useful for estimating the number of sialic acids contained in that cluster.

The vertical position at which each of the plurality of cluster indication marks 101 is placed in the mass spectrum corresponds to the total of the signal intensities of the peaks included in the isomer peak cluster corresponding to the indication mark 101 concerned. The larger the total of the signal intensities is, the higher the vertical position assigned to the cluster indication mark 101 is. Accordingly, in the example of FIG. 3, the cluster indication mark 101 corresponding to the second isomer peak cluster SIALIC #2 having the largest total of the signal intensities of the included peaks is displayed at the highest position in the vertical direction. By comparison, the cluster indication mark 101 corresponding to the third isomer peak cluster SIALIC #3 having the smallest total of the signal intensities of the included peaks is displayed at the lowest position in the vertical direction. The m/z range of the cluster indication mark 101 corresponding to the second isomer peak cluster SIALIC #2 and that of the cluster indication mark 101 corresponding to the third isomer peak cluster SIALIC #3 overlap each other. However, an overlap of the cluster indication marks 101 on the display can be avoided by changing the position of each indication mark 101 on the display in the previously described manner according to an order based on an intensity index, such as the total of the signal intensities of the peaks included in the isomer peak cluster.

When two or more kinds of sialic acids are set in the glycan search conditions, two or more isomer peak clusters which are identical in the number of sialic acids as well as in the glycan composition exclusive of the sialic acids and are only different in the kinds of sialic acids are considered to be isomer peak clusters which are related to each other. For example, consider the case where Neu5Ac and Neu5Gc are set as the kinds of sialic acids. As noted earlier, since their sugar residues have a mass difference of 16 Da, peaks which are adjacent to each other at an interval of 16 Da can be estimated as “different-sialic-acid-linked isomer peaks”, i.e., peaks which are identical in the number of sialic acids as well as in the glycan composition exclusive of the sialic acids and are only different in the kinds of sialic acids, which is either Neu5Ac or Neu5Gc. In that case, as shown in FIG. 3, arrow marks 102 are drawn from the vertical bars of the cluster indication mark 101 corresponding to the second isomer peak cluster SIALIC #2 having smaller m/z values to those of the cluster indication mark 101 corresponding to the third isomer peak cluster SIALIC #3 having larger m/z values. Furthermore, an annotation 103 is added to show that the mass difference Δm equals 16 Da, i.e., the mass difference between Neu5Ac and Neu5Gc. These elements visually help the user to easily understand the relationship between the peaks included in the second isomer peak cluster SIALIC #2 and those included in the third isomer peak cluster SIALIC #3.

The previously described task of detecting different-sialic-acid-linked isomer peaks does not always need to be performed in Step S5. It may be performed at any timing after isomer peak clusters have been detected in Step S3 and before the completion of the processing in Step S5.

The annotated mass spectrum creator 36 may create a peak table in place of, or along with, the annotated mass spectrum described earlier and display it on the screen of the display unit 5. The peak table is a table including a list which shows the m/z values of the included peaks for each isomer peak cluster detected in Step S3. The same table also describes the relationship of the corresponding peaks between the previously described, mutually related isomer peak clusters which are identical in the number of sialic acids as well as in the glycan composition exclusive of the sialic acids and are only different in the kinds of sialic acids.

FIG. 4 is a peak table corresponding to the annotated mass spectrum shown in FIG. 3. In this peak table 200, “Peak Index” is a figure (number) corresponding to the number of sialic acids of a specific linkage type. When there are a plurality of isomer peak clusters related to each other as clusters each having a product of the replacement of one kind of sialic acid by another, the peak indices of 1, 2, . . . are sequentially assigned, in ascending order of m/z value, to the peaks included in an isomer peak cluster including a peak having the smallest m/z value among all peaks in the plurality of isomer peak clusters related to each other. The same peak index is assigned to the peaks related to each other as clusters each having a product of the replacement of one kind of sialic acid by another, i.e., the peaks which are estimated to be identical in the number of sialic acids as well as in the glycan composition exclusive of the sialic acids and are only different in the kinds of sialic acids.

In the example of FIG. 4, the peak having the smallest m/z value among the peaks included in the second and third isomer peak clusters SIALIC #2 and SIALIC #3 which are related to each other is the peak at m/z 3038.1 in the second isomer peak cluster SIALIC #2. Therefore, Peak Index=1 is assigned to this peak. Peak Index=2 is assigned to the peak at m/z 3066.2 which is the second smallest m/z value in the second isomer peak cluster SIALIC #2. The peak with Peak Index=2 in the second isomer peak cluster SIALIC #2 is labelled as “2-2”.

In the third isomer peak cluster SIALIC #3 which is estimated to be composed of ions resulting from the replacement of N-acetylneuraminic acid by N-glycolylneuraminic acid, the peak index of the ion having the smallest m/z value of m/z 3082.2 is “2”, which is the same as the peak index of the corresponding peak in the second isomer peak cluster SIALIC #2, i.e., the peak from which the replacement of the sialic acid occurred. The “Relation” field in the peak table 200 shows additional information representing the relationship of the peaks between two isomer peak clusters related to each other. That is to say, the “Relation” field shows the serial number of the isomer peak cluster and the Peak Index, as well as the mass difference between the peaks, as the information representing the correspondence relationship of the different kinds of sialic acids on the peak-to-peak basis.

Both the annotated mass spectrum shown in FIG. 3 and the peak table shown in FIG. 4 allow the user to visually and immediately recognize the number of peaks included in each isomer peak cluster and the m/z value of each peak. The user can also easily understand the relationship between a plurality of isomer peak clusters which are identical in the number of sialic acids as well as in the glycan composition exclusive of the sialic acids and are only different in the kinds of sialic acids, and particularly, the relationship between the peaks included in those isomer peak clusters.

On the annotated mass spectrum or peak table thus displayed, the user selects and indicates one or more interesting isomer peak clusters by operating the input unit 4 (Step S6). For example, on the annotated mass spectrum, the user can point at a desired cluster indication mark 101 with the pointing device to select the isomer peak cluster corresponding to that indication mark 101. A similar selection can also be performed on the peak table by pointing at one of the numbers in the “SIALIC #” field.

The glycan-composition candidate table creator 37 responds to the selecting operation. For example, it collects glycan-composition candidates estimated in Step S4 for each peak included in the selected isomer peak cluster or clusters and displays a glycan-composition candidate table in which the collected candidates are related to each peak in the isomer peak clusters. The glycan-composition candidate table creator 37 may alternatively display a table of the glycan-composition candidates for all isomer peak clusters detected in Step S3. In this case, the glycan-composition candidates estimated for each peak included in the selected peak clusters can be highlighted in the table so that they are distinguishable from the other candidates. The display processor 38 displays the glycan-composition candidate table along with the annotated mass spectrum or peak table shown on the display unit 5 at the moment, in the same window or a separate window. It is also possible to display the glycan-composition candidate table by replacing the annotated mass spectrum or peak table with the candidate table, i.e., by switching the displayed content (Step S7).

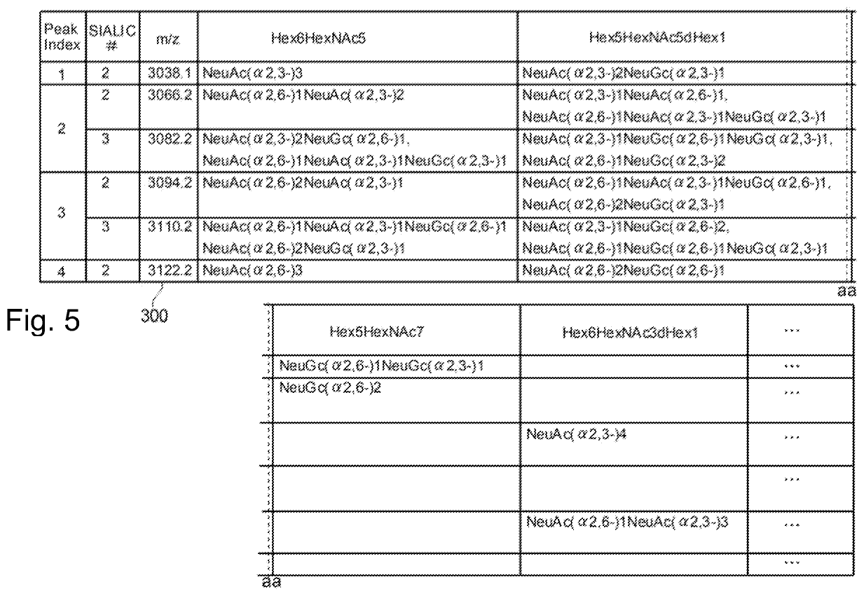

FIG. 5 is a glycan-composition candidate table 300 to be displayed when the second and third isomer peak clusters SIALIC #2 and SIALIC #3 have been selected in FIGS. 3 and 4. Actually, the upper and lower tables shown in FIG. 5 should be horizontally connected together at point “aa”. In other words, this table is actually a horizontally elongated table. Since the second isomer peak cluster SIALIC #2 and the third isomer peak cluster SIALIC #3 are related to each other, a glycan-composition candidate table corresponding to both clusters may automatically be created and displayed not only when both of the second and third isomer peak clusters SIALIC #2 and SIALIC #3 have been selected, but also when only one of those clusters SIALIC #2 and SIALIC #3 has been selected.

As shown in FIG. 5, in this glycan-composition candidate table 300, all peaks included in the two isomer peak clusters SIALIC #2 and SIALIC #3 are grouped by Peak Index, and each peak has an exhaustive list of the glycan-composition candidates estimated for that peak. The glycan-composition candidates are grouped so that the candidates which are identical in the glycan composition exclusive of the sialic acids appear in the same column.

Specifically, in the example of FIG. 5, one or more sialic-acid-containing glycans having three sialic acids and the glycan composition of Hex6HexNAc5 exclusive of the sialic acids, or one or more sialic-acid-containing glycans having three sialic acids and the glycan composition of Hex5HexNAC5dHex1 exclusive of the sialic acids, are estimated as glycan-composition candidates for any one of the peaks included in the two isomer peak clusters SIALIC #2 and SIALIC #3. Furthermore, sialic-acid-containing glycans having different glycan compositions (in the present example, Hex5HexNAc7 and Hex6HexNAc3dHex1) are also additionally estimated as glycan-composition candidates for some of those peaks.

One example is as follows. When the glycan composition exclusive of the sialic acids is assumed to be Hex6HexNAc5, the peak with Peak Index “2” (m/z 3082.2) in the third isomer peak cluster SIALIC #3 has two glycan-composition candidates estimated as the combination of the sialic-acid composition and linkage type, which are NeuAc(α2,3-)2NeuGc(α2,6-)1 and NeuAc(α2,6-)1NeuAc(α2,3-)1NeuGc(α2,3-)1. Whether this ion peak has originated from the mixture of these two kinds of glycans or from only one of them can be determined by performing an MS/MS analysis in which that ion peak is selected as the precursor ion, as will be described later.

In the annotated mass spectrum or peak table, when an isomer peak cluster is selected and indicated by the user, the character string corresponding to the selected isomer peak cluster, or those corresponding to the peaks belonging or related to that cluster, may be highlighted so that the cluster or peaks will be visually noticeable in the annotated mass spectrum or peak table.

Regardless of whether or not a plurality of glycan-composition candidates have been estimated for a peak, when an operation for indicating a desired peak as the precursor ion is performed by the user in the annotated mass spectrum, peak table or glycan-composition candidate table, the precursor ion selection receiver 39 selects the indicated ion peak as the precursor ion for an MS/MS analysis (Step S8).

The information of this selection is sent to the analysis control unit 2. The analysis control unit 2 operates the mass spectrometry unit 1 so as to perform an MS/MS analysis, or more specifically, a product ion scan measurement employing an ion dissociation technique, such as collision induced dissociation, with the selected precursor ion as the target. The mass spectrometry unit 1 performs an MS/MS analysis on a sample containing glycans which have undergone the sialic-acid-linkage specific modification, to obtain MS/MS spectrum data (Step S9).

In the MS/MS spectrum, a plurality of product ion peaks originating from the targeted sialic-acid-containing glycan are observed. Based on the m/z values of those peaks, the user can determine which of the plurality of glycan-composition candidates is the most plausible candidate, or whether or not the single glycan-composition candidate is an appropriate candidate (Step S10).

As described thus far, a structural analysis of sialic-acid-containing glycans, including the linkage type of sialic acids, can be efficiently performed by the glycan analyzing system according to the present embodiment.

In the description of the previous embodiment, all glycan-composition candidates estimated for each peak in Step S4 are shown in the glycan-composition candidate table 300 as shown in FIG. 5. It is also possible to further narrow down the glycan-composition candidates according to specific constraints or assumptions and display all candidates in such a manner that the glycan-composition candidates selected by the narrowing-down process can be visually and easily distinguished from those excluded from the selection. In that case, the glycan composition filter 35 evaluates each glycan-composition candidate according to previously set narrowing conditions, and the glycan-composition candidate table creator 37 changes the display of the glycan-composition candidates based on the result of the narrowing-down process.

The narrowing-down conditions in the glycan composition filter 35 can be appropriately determined. For example, the conditions described in Patent Literature 1 can be applied. In particular, when there is a considerable number of glycan-composition candidates, the burden of the user to examine the candidates can be reduced by displaying those candidates in such a manner that more plausible candidates are distinguished from less likely ones.

If an ion peak originating from a glycan which should be observed has not been detected in Step S2 for some reason, such as the signal intensities of the peaks being low on the entire basis, the isomer peak clusters will not also be appropriately detected, which may cause some problems in the estimation of the glycan composition. In such a case, for example, the user can modify the peak detection conditions so that a greater number of monoisotopic ion peaks will be detected in Step S2. Such an modification may possibly result in an appropriate estimation of the glycan composition.

It should be noted that the previous embodiment is a mere example of the present invention. Any change, modification or addition appropriately made within the gist of the present invention will naturally be included within the scope of claims of the present application.

### VARIOUS MODES

A person skilled in the art can understand that the previously described illustrative embodiment is a specific example of the following modes of the present invention.

(Clause 1) One mode of the method for analyzing a sialic-acid-containing glycan according to the present invention is an analysis method for analyzing a sample containing a sialic-acid-containing glycan including a modification specific to a sialic-acid linkage type, or a sample containing a molecule modified with the same glycan, based on mass spectrum data obtained by a mass spectrometric analysis of the sample, the method including:

a peak detection step for detecting, from the mass spectrum data, a representative peak for each isotope peak cluster;

a peak cluster detection step for detecting, from representative peaks detected in the peak detection step, an isomer peak cluster including a plurality of ion peaks estimated to be identical in the number of sialic acids and in the glycan composition exclusive of the sialic acids;

a composition estimation step for estimating a glycan composition for a representative peak detected in the peak detection step, according to a predetermined glycan search condition;

a first display process step for creating an annotated mass spectrum in which an annotation is added for each isomer peak cluster detected in the peak cluster detection step to indicate the correspondence between each peak included in one isomer peak cluster and a peak observed in a mass spectrum, or creating a peak table which shows, for each isomer peak cluster detected in the peak cluster detection step, the mass-to-charge-ratio values of the peaks included in one isomer peak cluster, and for displaying the mass spectrum or the peak table on a display section; and

a second display process step for creating a composition candidate table in which each glycan-composition candidate obtained in the composition estimation step is related to at least one isomer peak cluster detected in the peak cluster detection step, and for displaying the composition candidate table on the display section along with or in a switchable manner with the annotated mass spectrum or the peak table.

(Clause 5) One mode of the device for analyzing a sialic-acid-containing glycan according to the present invention is an analyzing device for analyzing a sample containing a sialic-acid-containing glycan including a modification specific to a sialic-acid linkage type, or a sample containing a molecule modified with the same glycan, based on mass spectrum data obtained by a mass spectrometric analysis of the sample, the device including:

a peak detector configured to detect, from the mass spectrum data, a representative peak for each isotope peak cluster;

a peak cluster detector configured to detect, from representative peaks detected by the peak detector, an isomer peak cluster including a plurality of ion peaks estimated to be identical in the number of sialic acids and in the glycan composition exclusive of the sialic acids;

a composition estimator configured to estimate a glycan composition for a representative peak detected by the peak detector, according to a predetermined glycan search condition;

a display processor configured to create an annotated mass spectrum in which an annotation is added for each isomer peak cluster detected by the peak cluster detector to indicate the correspondence between each peak included in one isomer peak cluster and a peak observed in a mass spectrum, or to create a peak table which shows, for each isomer peak cluster detected by the peak cluster detector, the mass-to-charge-ratio values of the peaks included in one isomer peak cluster, and further configured to create a composition candidate table in which each glycan-composition candidate obtained by the composition estimator is related to at least one isomer peak cluster detected by the peak cluster detector, and to display the composition candidate table on a display section along with or in a switchable manner with the annotated mass spectrum or the peak table.

In the method for analyzing a sialic-acid-containing glycan according to Clause 1 and the device for analyzing a sialic-acid-containing glycan according to Clause 5, the user can intuitively recognize a plurality of peaks included in each isomer peak cluster by referring to the annotated mass spectrum or peak table shown on the display section. Subsequently, the user can refer to the composition candidate table and conveniently recognize, for each of the peaks included in one or more isomer peak clusters, the glycan-composition candidates corresponding to the peak, and determine, for example, a precursor ion for an MS/MS analysis necessary for verifying indeterminate glycan compositions. This improves the efficiency of the structural analysis of sialic-acid-containing glycans. It can also lower the probability of manual errors in the analyzing task, thereby enhancing the analysis accuracy.

(Clauses 2 and 6) In the method for analyzing a sialic-acid-containing glycan according to Clause 1 and the device for analyzing a sialic-acid-containing glycan according to Clause 5, the annotated mass spectrum and the peak table may include information showing the correspondence of peaks which are identical in the linkage type of sialic acids among a plurality of isomer peak clusters which are identical in the number of sialic acids as well as in the glycan composition exclusive of the sialic acids and are only different in the kinds of sialic acids.

Typical examples of the kinds of sialic acids include N-acetylneuraminic acid, N-glycolylneuraminic acid and deaminated neuraminic acid.

By using the method for analyzing a sialic-acid-containing glycan according to Clause 2 and the device for analyzing a sialic-acid-containing glycan according to Clause 6, the user can easily understand the correspondence of the peaks among a plurality of isomer peak clusters which are only different in the kinds of sialic acids. For example, the user can conveniently recognize the presence or absence of a specific phenomenon which can occur in living organisms, such as a replacement of N-acetylneuraminic acid by N-glycolylneuraminic acid.

(Clause 3) The method for analyzing a sialic-acid-containing glycan according to Clause 1 or 2 may further include:

a precursor ion selection step for receiving an operation by a user for selecting, as a precursor ion, one of the peaks included in one of the isomer peak clusters in the annotated mass spectrum, the peak table or the composition candidate table shown on the display section; and

an MS/MS analysis execution step for executing an MS/MS analysis on the sample, with the precursor ion selected in the precursor ion selection step as the target.

(Clause 7) The device for analyzing a sialic-acid-containing glycan according to Clause 5 or 6 may further include:

a precursor ion selection receiver configured to receive an operation by a user for selecting, as a precursor ion, one of the peaks included in one of the isomer peak clusters in the annotated mass spectrum, the peak table or the composition candidate table shown on the display section; and

an MS/MS analysis executer configured to execute an MS/MS analysis on the sample, with the precursor ion received by the precursor ion selection receiver as the target.

In the precursor ion selection step, the peak to be designated as a precursor ion can be selected by a convenient operation, such as a click of a pointing device on a screen of the display section.

In general, when a plurality of glycan-composition candidates have been estimated for one peak, it is necessary to perform an MS/MS analysis for that peak and examine the mass-to-charge ratios of the resulting product ions in order to determine which glycan-composition candidate is the correct candidate. The analysis method according to Clause 3 and the analyzing device according to Clause 7 allows the user to understand, for example, which peak in the composition candidate table needs an MS/MS analysis, and to execute an MS/MS analysis with the ion corresponding to that peak designated as the precursor ion by a convenient operation. Therefore, the structural analysis of sialic-acid-containing glycans can be even more efficiently performed.

(Clause 4) The method for analyzing a sialic-acid-containing glycan according to one of Clauses 1-3 may further include a peak-detection-condition resetting step for receiving an operation by a user for modifying a peak detection condition after the annotated mass spectrum, the peak table or the glycan-composition candidate table is displayed, wherein the analysis is once more carried out by performing the peak detection step and the subsequent steps under the modified peak detection condition.

By the analysis method according to Clause 4, the user can once more perform an analysis after appropriately modifying the peak detection condition when, for example, no isomer peak cluster can be detected due to some factors, such as low signal intensities or insufficient signal-to-noise ratios of the peaks observed in the mass spectrum. Therefore, the efficiency of the glycan analysis can be improved even when the condition of the sample is unfavorable.

### REFERENCE SIGNS LIST

### 1 . . . Mass Spectrometry Unit

### 2 . . . Analysis Control Unit

### 3 . . . Data Analysis Unit

### 30 . . . Data Storage Section

### 31 . . . Peak Detector

### 32 . . . Glycan Search Condition Setter

### 320 . . . Glycan Search Condition Storage Section

### 33 . . . Isomer Peak Cluster Detector

### 34 . . . Glycan Composition Estimator

### 35 . . . Glycan Composition Filter

### 36 . . . Annotated Mass Spectrum Creator

### 37 . . . Glycan-Composition Candidate Table Creator

### 38 . . . Display Processor

### 39 . . . Precursor Ion Selection Receiver

### 4 . . . Input Unit

### 5 . . . Display Unit

1. A method for analyzing a sample containing a sialic-acid-containing glycan including a modification specific to a sialic-acid linkage type, or a sample containing a molecule modified with the same glycan, based on mass spectrum data obtained by a mass spectrometric analysis of the sample, the method comprising:
a peak detection step for detecting, from the mass spectrum data, a representative peak for each isotope peak cluster;
a peak cluster detection step for detecting, from representative peaks detected in the peak detection step, an isomer peak cluster including a plurality of ion peaks estimated to be identical in a number of sialic acids and in a glycan composition exclusive of the sialic acids;
a composition estimation step for estimating a glycan composition for a representative peak detected in the peak detection step, according to a predetermined glycan search condition;
a first display process step for creating an annotated mass spectrum in which an annotation is added for each isomer peak cluster detected in the peak cluster detection step to indicate a correspondence between each peak included in one isomer peak cluster and a peak observed in a mass spectrum, or creating a peak table which shows, for each isomer peak cluster detected in the peak cluster detection step, mass-to-charge-ratio values of the peaks included in one isomer peak cluster, and for displaying the mass spectrum or the peak table on a display section; and
a second display process step for creating a composition candidate table in which each glycan-composition candidate obtained in the composition estimation step is related to at least one isomer peak cluster detected in the peak cluster detection step, and for displaying the composition candidate table on the display section along with or in a switchable manner with the annotated mass spectrum or the peak table.

2. The method for analyzing a sample containing a sialic-acid-containing glycan according to claim 1, wherein the annotated mass spectrum and the peak table include information showing a correspondence of peaks which are identical in the linkage type of sialic acids among a plurality of isomer peak clusters which are identical in the number of sialic acids as well as in the glycan composition exclusive of the sialic acids and are only different in kinds of sialic acids.

3. The method for analyzing a sample containing a sialic-acid-containing glycan according to claim 1, further comprising:
a precursor ion selection step for receiving an operation by a user for selecting, as a precursor ion, one of the peaks included in one of the isomer peak clusters in the annotated mass spectrum, the peak table or the composition candidate table shown on the display section; and
an MS/MS analysis execution step for executing an MS/MS analysis on the sample, with the precursor ion selected in the precursor ion selection step as a target.

4. The method for analyzing a sample containing a sialic-acid-containing glycan according to claim 1, further comprising a peak-detection-condition resetting step for receiving an operation by a user for modifying a peak detection condition after the annotated mass spectrum, the peak table or the glycan-composition candidate table is displayed,
wherein the analysis is once more carried out by performing the peak detection step and subsequent steps under the modified peak detection condition.

5. A device for analyzing a sample containing a sialic-acid-containing glycan including a modification specific to a sialic-acid linkage type, or a sample containing a molecule modified with the same glycan, based on mass spectrum data obtained by a mass spectrometric analysis of the sample, the device comprising:
a peak detector configured to detect, from the mass spectrum data, a representative peak for each isotope peak cluster;
a peak cluster detector configured to detect, from representative peaks detected by the peak detector, an isomer peak cluster including a plurality of ion peaks estimated to be identical in a number of sialic acids and in a glycan composition exclusive of the sialic acids;
a composition estimator configured to estimate a glycan composition for a representative peak detected by the peak detector, according to a predetermined glycan search condition;
a display processor configured to create an annotated mass spectrum in which an annotation is added for each isomer peak cluster detected by the peak cluster detector to indicate a correspondence between each peak included in one isomer peak cluster and a peak observed in a mass spectrum, or to create a peak table which shows, for each isomer peak cluster detected by the peak cluster detector, mass-to-charge-ratio values of the peaks included in one isomer peak cluster, and further configured to create a composition candidate table in which each glycan-composition candidate obtained by the composition estimator is related to at least one isomer peak cluster detected by the peak cluster detector, and to display the composition candidate table on a display section along with or in a switchable manner with the annotated mass spectrum or the peak table.

6. The device for analyzing a sample containing a sialic-acid-containing glycan according to claim 5, wherein the annotated mass spectrum and the peak table include information showing a correspondence of peaks which are identical in the linkage type of sialic acids among a plurality of isomer peak clusters which are identical in the number of sialic acids as well as in the glycan composition exclusive of the sialic acids and are only different in kinds of sialic acids.

7. The device for analyzing a sample containing a sialic-acid-containing glycan according to claim 5, further comprising:
a precursor ion selection receiver configured to receive an operation by a user for selecting, as a precursor ion, one of the peaks included in one of the isomer peak clusters in the annotated mass spectrum, the peak table or the composition candidate table shown on the display section; and
an MS/MS analysis executer configured to execute an MS/MS analysis on the sample, with the precursor ion received by the precursor ion selection receiver as a target.
